(19) United States
(12) Patent Application Publication
(10) Pub. No.: US 2025/0009480 A1
(43) Pub. Date: Jan. 9, 2025

(54) BONE REGENERATION SYSTEM

(71) Applicants: BIOTECH DENTAL, Salon-de-Provence (FR); THE BONE INSTITUTE, NICE (FR)

(72) Inventor: Jérôme SURMENIAN, NICE (FR)

(21) Appl. No.: 18/765,138

(22) Filed: Jul. 5, 2024

(30) Foreign Application Priority Data
Jul. 7, 2023 (FR) 2307269

(51) Int. Cl. A61C 8/02; A61C 8/00

(52) U.S. Cl. CPC A61C 8/0006; A61C 8/0012; A61C 8/0027; A61C 8/0031; A61C 8/0089

(57) ABSTRACT

The present invention relates to a device intended to ensure the maintenance of a bone regeneration substitute (and if necessary of a cover membrane), on a bone defect of a moderate size in a jaw bone of a patient comprising a single piece formed by (1) an external pillar which is intended to be fixed in the anchor foot within the jaw bone, which external pillar comprises anti-rotational means making it possible to inhibit the relative movements of the external pillar relative to the anchor foot and by (2) a cover piece to be placed on the bone regeneration substitute so as to delimit the volume of bone regeneration at said bone defect site in combination with said bone regeneration substitute said cover piece presenting a central part in the continuation of the external pillar, and at least 2 flexible elongated lateral extensions in the continuation of the central part, which does not comprise any orifice intended for positioning of a fixing screw, and whose axis intersects perpendicularly the central axis (AA), corresponding to the axis crossing the central part and the anchor foot.

The present patent application claims the priority of the French patent application FR2307269, filed on Jul. 7, 2023, which is incorporated herein by reference.

### FIELD OF INVENTION

The present invention relates to the field of devices intended for bone regeneration and, more specifically, to that of the stabilization of endosseous dental implants.

### PRIOR ART

Classically, endosseous implants take the form of an anchor foot adapted for implantation in a patient's jaw bone, which anchor foot most often corresponds to an anchor screw.

An example of such implants is given in patent application US 2003/232308 in which an anchor foot (base) intended to be positioned in a housing of complementary shape prepared in the jaw bone of the patient also presents a collar (platform) extending from the base and with which it therefore forms a single piece. Now, this anchor foot most often cooperates with an external pillar which is intended to be fixed in the anchor foot and which will be coupled to a prosthetic connection allowing a crown to be fixed to replace the missing dental element.

Now, such implantation can be complicated by the presence of bone defects within the patient's jaw itself. Such bone defects, which can have multiple origins (infectious, tumoral or traumatic), result from a lack of bone healing and correspond to cavities in place of the destroyed bone.

Faced with such bone defects, it is possible to use biomaterials allowing both the filling of the bone defect and promoting bone healing within it.

Typically, the biomaterial used consists of either synthetic bone substitute material (e.g., hydroxyapatite granules) or bone particles. Once the recess corresponding to the bone defect filled with this biomaterial, this recess is closed by a cover membrane or plate which makes it possible to direct and contain the growth of this biomaterial, we speak of Guided Bone Regeneration (GBR). As an example of such cover membranes and plates, we can cite those described in U.S. Pat. No. 4,816,339 and in patent EP 2,536,446 B1 respectively.

Each of these solutions (membrane or covering plate) has its respective advantages and disadvantages.

The cover membrane, by its structure, allows the passage of nutrients allowing bone regeneration while not being colonized by bone cells. Also, the cover membrane can simply be removed once regeneration is complete without risking weakening or damaging the regenerated bone. Now, the “flexible” nature of this cover membrane makes it difficult to control the volume of bone regeneration.

Conversely, the cover plate, due to its rigidity, makes it possible to optimally guide the volume of this regeneration. Now, the perforation of its structure to allow the passage of nutrients towards the regeneration site leads to its colonization by bone cells. As a result, removal of the cover plate at the end of regeneration is complicated and is likely to affect the integrity of the regenerated bone.

As for maintaining the cover membrane or plate, it is ensured by screws fixed to the bone surrounding the bone defect.

Now, and in the case of a bone defect of moderate size, the practitioner will generally use a cover membrane. This cover membrane can be maintained by a screw fixing the anchor foot of the future dental implant prepositioned within the bone defect of the patient's jaw. Now, the practitioner will not use any cover plate, since such cover plate necessitate multiple fixation points each generating a bone trauma. Thus, these bone traumas are bone defects that can be greater than the one, whose generation is ambitioned.

Also, it is important to develop new solutions for bone regeneration with regard to bone defects of moderate size specifically, which present simplified implementation for the practitioner, minimal trauma to the jaw bone of the patient, while ensuring the best possible result in terms of bone regeneration at the bone defect site.

### SUMMARY OF THE INVENTION

Faced with this problem, the inventor has now developed a new solution adapted to the bone regeneration of bone defects of moderate size which, apart from the fixation associated with the anchor foot of the implant to come, does not require any fixation in the bone surrounding the bone defect, while framing the volume of bone regeneration in an improved manner. This particularly innovative solution may or may not use a cover membrane.

In detail, a first object of the invention relates to a bone regeneration kit for a bone defect site, preferably of a moderate size, in a jaw bone of a patient which comprises:
1) a bone regeneration substitute allowing the filling of the bone defect and
2) a bone regeneration system comprising:
2a) at least one anchor foot suitable for endosteal installation in the jaw bone, preferably within the bone defect itself;
2b) optionally at least one cover membrane, preferably a cover membrane whose surface makes it possible to cover at least the entire bone defect filled by the bone regeneration substitute; and
3) at least one device intended to ensure the maintenance of the bone regeneration substitute and, optionally of the cover membrane, on said bone defect, which device comprises:
3-1) at least one external pillar on its face directed towards the bone, which is intended to be fixed in the anchor foot, which external pillar comprises anti-rotational means making it possible to inhibit the relative movements of the external pillar relative to the anchor foot (3) once the external pillar placed in the anchor foot; and
3-2) at least one cover piece on its face opposite the bone, which is placed on the bone regeneration substitute and, optionally, on a cover membrane, so as to delimit the volume of bone regeneration at said bone defect site in combination with said bone regeneration substitute and, optionally, said cover membrane, which cover piece forms a single piece with the external pillar and presents:
3-2.i) a central part, in the continuation of the external pillar, and
3-2.ii) at least 2 flexible elongated lateral extensions in the continuation of this i) central part, which does not comprise any orifice intended for positioning of a fixing screw, and whose axis intersects perpendicularly the central axis (AA), corresponding to the axis crossing the central part and the anchor foot.
3a) on its face directed towards the bone, at least one external pillar intended to be fixed in the anchor foot, which external pillar comprises anti-rotational means making it possible to inhibit the relative movements of the external pillar by relation to the anchor foot once the external pillar is placed in the anchor foot; and
3b) on its face opposite the bone, at least one cover piece placed on the bone regeneration substitute and, optionally, on the cover membrane so as to delimit, in combination with said bone regeneration substitute and, optionally, said cover membrane, the volume of bone regeneration at said bone defect site, which cover piece has:
3bi) a central part, in the extension of the external pillar, and
3bii) at least 2 elongated lateral extensions in the continuation of this i) central part whose axis intersects perpendicularly the central axis (AA), corresponding to the axis crossing the central part and the 1) at least one anchor pillar, preferably each elongated lateral extension does not include any orifice intended for positioning a fixing screw and/or said elongated lateral extensions are flexible.

A second object of the invention relates to a bone regeneration system as described above.

A third object of the invention relates to a device as defined previously intended for maintaining the bone regeneration substitute and, possibly, the cover membrane.

According to a preferred embodiment, the device intended to ensure the maintenance of the bone regeneration substitute and, possibly, of the cover membrane on the bone defect comprises an intermediate pillar, between the external pillar and the cover piece. Preferably, the cover piece forms a single part with the external pillar and the intermediate pillar.

According to another preferred embodiment, the system according to the invention further comprises at least one screw which having a head and an external thread at its distal end, wherein the at least one external pillar of the device, and, if present the at least one intermediate pillar, has an internal cavity allowing the passage of said the at least one screw but not of its head; and wherein the at least one anchor foot of the system has a cavity in which is provided a tapping, which can cooperate with the thread of said at least one screw to allow the assembly of the at least one anchor foot and of the at least one device.

According to a complementary preferred embodiment, the system according to the invention further comprises 4) at least one piece facilitating the griping of the device by the practitioner (when putting the device in place), taking the form of a sleeve, and which clips to the central part of the device in a complementary housing located within this central part.

A fourth object relates to a method of bone regeneration for a bone defect site of moderate size in a jaw bone of a patient, which method comprises the steps of:
i. placement of an anchor foot within the patient's jaw bone at or near the bone defect;
ii. filling of the bone defect with a bone regeneration substitute;
iii. eventually positioning, on this bone regeneration substitute, a cover membrane; and
iv. placement, preferably on this cover membrane if positioned, a device as defined previously intended to ensure the maintenance of the bone regeneration substitute and, possibly, the cover membrane.

### DESCRIPTION OF FIGURES

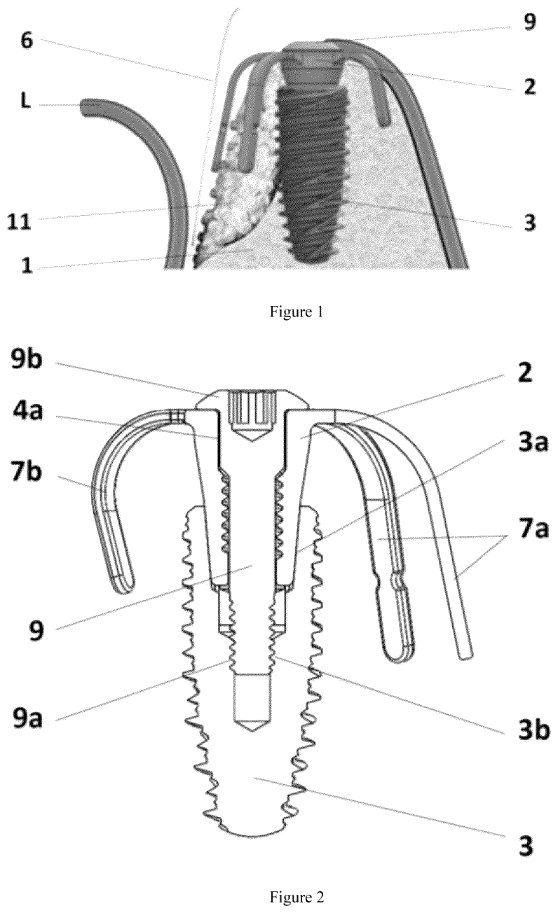

The FIG. 1 is a sectional view of the implantation in the jaw bone of a patient of an embodiment of the bone regeneration system according to the invention.

The FIG. 2 is a sectional view of this same embodiment of the bone regeneration system according to the invention.

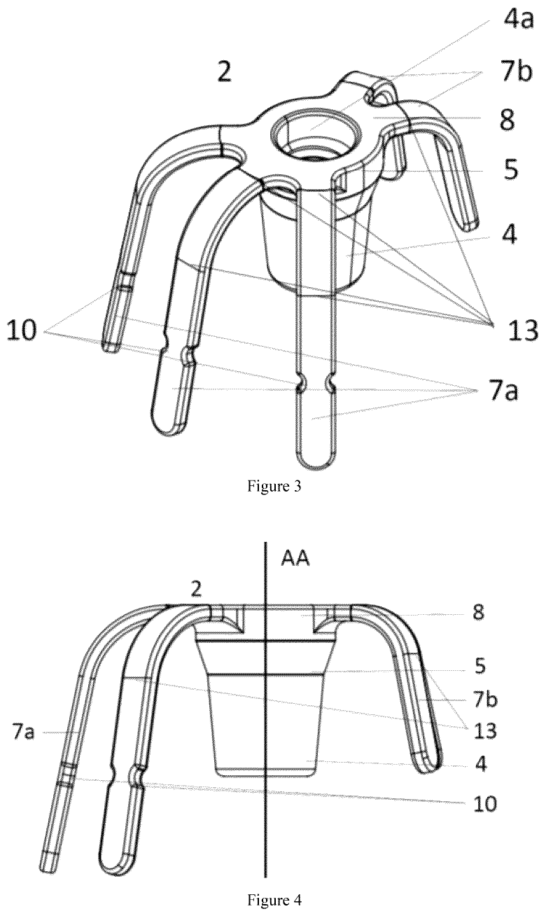

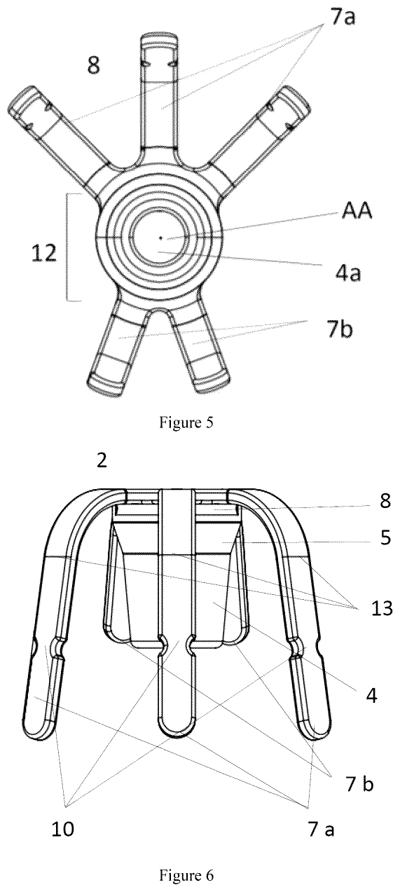

The FIG. 3, FIG. 4, FIG. 5 and FIG. 6 show schematic views from different angles of one embodiment of a device according to the invention intended to ensure the maintenance of the bone regeneration substitute.

### DETAILED DESCRIPTION OF THE INVENTION

Bone regeneration substitutes are well known to those skilled in the art and such a substitute can be chosen from those used for filling bone loss in various pathologies. Such a bone regeneration substitute may consist of a synthetic bone substitute material (for example hydroxyapatite granules), but also of bone particles. As an example of bone regeneration substitutes, we can cite RE-BONE, BIO-OSS, BIOSORB-DENTAL, CREOS, ADBONE, TEEBONE, SYBONE, etc.

Concerning the anchor feet, these are also well known to practitioners and typically take the form of a screw capable of being screwed into a complementary housing prepared and shaped in the patient's maxillary bone. The patient's jaw bone is thus tapped to obtain a thread which will then allow the anchor foot to be fixed. The anchor foot screw may be cylindrical or conical in shape.

The material of this anchor foot is advantageously compatible with the biological material of the bone so as to facilitate its osseointegration in the jaw (maxillary and mandibular bone). Typically, this anchor foot can be made of zirconia (ceramic), titanium or even a titanium alloy.

In terms of dimensions, such an anchor foot has:
a length of between 0.2 and 1.5 cm, preferably between 0.5 and 0.8 cm; and
a section between 3 and 8 mm, preferably between 3 and 6 mm.

Cover membranes have long been used in Guided Bone Regeneration (GBR) and are, in fact, well known to practitioners. They can be resorbable or non-resorbable in nature.

The cover membranes conventionally used are non-resorbable and are made from ePTFE (expanded polytetrafluoroethylene) with the possibility of mechanical reinforcement with a metal frame; typically made of titanium (ePTFE TR for Titanium Reinforced).

Resorbable membranes can be either synthetic or collagenous.

Synthetic membranes are typically made of copolymers of polylactic and polyglycolic acid, which is why they are resorbed by hydrolysis. Examples of such synthetic membranes include membranes marketed under the trade names ATRISORB, VICRYL, RESOLUT TM/RESOLUT XT.

Collagenous membranes were first used in 1996 and are made from cross-linked or non-cross-linked collagen which is of bovine or porcine origin with cross-linked or non-cross-linked fibers. Examples of such collagen membranes include membranes marketed under the trade names AVITENE, PAROGUIDE, BIO-GUIDE, PERIOGEN or even BIOMEND.

The dimensions of the at least one cover membrane are such that it should preferably make it possible to cover at least the entire bone defect.

Typically, such a cover membrane presents:
a length of between 0.5 and 2.5 cm, preferably between 0.5 and 1.5 cm; and
a width of between 0.5 and 2.5 cm, preferably between 0.5 and 1.5 cm.

The device intended to ensure the maintenance of the bone regeneration substitute and, possibly, of the cover membrane on the bone defect must have sufficient rigidity so as to ensure its function. Now and preferably, the cover piece of this same device must simultaneously present an elasticity which allows its adjustment, during its installation, by the curvature of its elongated lateral extensions, so as to delimit, possibly in combination with the cover membrane, the volume of regeneration of the bone defect.

To do this, the device ensuring the maintenance of the bone regeneration substitute and possibly the cover membrane, or at least its cover piece, is advantageously made of titanium.

Titanium is a shiny transition metal with low density and high strength. Pure titanium is therefore stronger than ordinary low-carbon steels, while being 45% lighter. The integration of titanium into different alloys allows them to be associated with particularly interesting properties. Thus, titanium alloys are generally used for most applications with small amounts of aluminum and vanadium, typically 6% and 4% respectively, by weight. Titanium can be found in two crystalline forms (alpha and beta) and, titanium alloys, depending on their composition, will orient the titanium towards one or the other of these crystalline forms, thereby modifying the properties of the alloy. Among these alloys, Grade 1 titanium, or T35, and Grade 2 titanium, or T40, constitute specific alloys, whose composition (in Titanium, Oxygen, Iron, Carbon, Hydrogen and Nitrogen) and the characteristics are well known to those skilled in the art.

Advantageously, the device ensuring the maintenance of the bone regeneration substitute and, possibly, of the cover membrane is made of grade 1 or grade 2 titanium.

According to a preferred embodiment, the 3) at least one device intended to ensure the maintenance of the A) bone regeneration substitute and, optionally, the 2) at least one cover membrane on the bone defect comprises, between 3a) at least one external pillar and 3b) at least one cover piece, 3c) at least one intermediate pillar which could be described as a transmucosal part.

Ideally, the height of this 3c) at least one intermediate pillar is in the range from 0.5 to 5 mm, preferably in the range from 1 to 2 mm.

Preferably, this 3c) at least one intermediate pillar comprises a constriction in the width direction going towards the jaw bone and may, possibly, also comprise a festoon. This constriction ideally has a conical profile, which conical profile is devoid of any potentially harmful angle.

According to another preferred embodiment, the system according to the invention further comprises:
and for the 3) at least one device ensuring the maintenance of the bone regeneration substitute and, optionally, the cover membrane, 3d) at least one screw which has, at its distal end, an external thread, and provided within said a) at least one external pillar and, if present, within said 3c) at least one intermediate pillar, an internal cavity allowing the passage of said 3d) at least one screw but not of its head; and
within said 1) at least one anchor foot, a cavity in which is provided a tapping which can cooperate with the thread of the 3d) at least one screw to allow the assembly of said 1) at least one anchor foot and 3) at least one device ensuring the maintenance of the bone regeneration substitute and, optionally, the cover membrane by screwing the thread of said 3d) at least one screw in the tapping of the internal cavity of 1) at least one anchor foot.

In this embodiment, this 3d) at least one screw allows the assembly by trans-screwing of said 1) at least one anchor foot and 3) at least one device ensuring the maintenance of the bone regeneration substitute and, possibly, the cover membrane.

Advantageously, the head of said 3d) at least one screw and 3a) at least one external pillar or, if present, 3c) at least one intermediate pillar have centering means. Typically, these centering means are such that the head of said d) at least one screw and said 3a) at least one external pillar or, if present, said 3c) at least one intermediate pillar comprise female and male complementary portions. Preferably, these complementary female and male shapes are frustoconical shapes with a vertical axis. Therefore, in addition to centering, these shapes make it possible to inhibit relative movements and ensure sealing between the d) at least one screw and the a) at least one external pillar or, if present, the c) at least one intermediate pillar.

Preferably, the d) at least one screw and the a) at least one external pillar or, if present, the 3c) at least one intermediate pillar, have complementary centering means which are included in the length of the 3d) at least one screw and in the internal cavity of said 3a) at least one external pillar or, if present, of said 3c) at least one intermediate pillar.

Again advantageously, a tapping is provided within the internal cavity of a) at least one external pillar. This tapping can cooperate with an external thread present, for example on an extractor, so as to allow the disassembly of 1) at least one anchor foot of 3) at least one device intended to ensure the maintenance of the bone regeneration substitute and, possibly, of the cover membrane on said bone defect.

Now linked to 3b) at least one cover piece, this has 3bi) a central part, in the continuation of 3a) at least one external pillar or, if present, from 3c) to less an intermediate pillar, which 3bi) central part is advantageously circular and then typically takes the shape of a washer.

This 3b) at least one cover piece furthermore has, 3bii) at least 2 elongated lateral extensions, preferably 3bii) at least 3 extensions, which elongated lateral extensions are advantageously flexible.

Also, the 3bi) central part of the 3b) at least one cover piece constitutes the base of the 3bii) at least 2 elongated lateral extensions, preferably 3bii) at least 3 elongated lateral extensions.

The elongated lateral extensions have sufficient length and width to allow their positioning on the bone regeneration substitute and, possibly, the cover membrane and to ensure their maintenance. Typically, the elongated lateral extensions have a length of between 3 and 15 mm, preferably between 3 and 10 mm. Typically still, the elongated lateral extensions have a width of between 1 and 8 mm, preferably between 2 and 5 mm.

Advantageously, and to facilitate the adaptation to each patient of the device intended to ensure the maintenance of the bone regeneration substitute and, possibly, of the cover membrane, each elongated lateral extension may have one or more predetermined break points positioned in its length. In this way, and when installing the device by the practitioner, the latter will be able to use one or more of these predetermined breaking points, so as to shorten all or part of these elongated lateral extensions and to adapt them to the patient's morphology. Following this shortening, the practitioner can, simply and using the instruments at his disposal, eliminate the angles of the break point(s) used, so as not to risk injuring the patient.

Advantageously again, and always to facilitate the adaptation to each patient of the device intended to ensure the maintenance of the bone regeneration substitute and, possibly, of the cover membrane, each elongated lateral extension may have one or more primers which facilitate its folding, and which are positioned along its length. In this way, and when the device is fitted by the practitioner, he will be able to use one or more of these primers, so as to fold all or part of these elongated lateral extensions and adapt them to the morphology of the patient.

The 3bi) central part of the 3b) at least one cover piece has two distinct halves corresponding to a vestibular half and to a palatal half corresponding to the half facing the vestibular side (i.e., towards the outside the mouth) and the half facing the palatal side (i.e. towards the palate) respectively.

Advantageously, the elongated lateral extensions in the continuation of the vestibular half of the central part, or elongated vestibular lateral extensions, have a greater length than the elongated lateral extensions in the continuation of the palatal half of the central part or elongated palatal lateral extensions.

This characteristic makes it easier for the practitioner to differentiate between the vestibular side and the palatal side of the device intended to ensure the maintenance of the bone regeneration substitute and, possibly, of the cover membrane.

Typically, the elongated vestibular lateral extensions have a length of between 4 and 10 mm.

Advantageously, there will be 2 to 5 elongated vestibular lateral extensions, preferably 3 to 4 and, particularly preferably, 3 elongated vestibular lateral extensions.

Ideally, the elongated vestibula lateral extensions have the same spacing between them. Typically, said spacing is at least 10°, preferably at least 20° and, particularly preferably, at least 25°.

As for the elongated palatal lateral extensions, their length is typically between 3 and 6 mm.

Advantageously, there will be from 1 to 4 elongated palatal lateral extensions, preferably 2 to 3 and, particularly preferably, 2 elongated palatal lateral extensions.

Ideally, the elongated palatal lateral extensions have the same spacing between them. Typically, said spacing is at least 10°, preferably at least 20° and, particularly preferably, at least 25°.

To present these particularly advantageous properties:
the 3bi) at least one central part of the 3b) at least one cover piece of said 3) at least one device according to the invention has a thickness of between 0.3 and 3 mm, preferably between 0.5 and 1.5 mm.
the elongated lateral extensions have a thickness of between 0.1 and 1.5 mm, preferably between 0.3 and 1 mm.

According to another preferred embodiment, the 3b) at least one cover piece forms a single part with the 3a) at least one external pillar and, optionally, the 3c) at least one intermediate pillar. To do this, these can be produced simultaneously as a single piece or be coupled subsequently, in particular by welding or crimping.

Production methods suitable for the manufacture of the 3b) at least one cover piece with 3a) at least one external pillar and/or with 3c) at least one intermediate pillar as a single piece are well known to those skilled in the art. Examples of such methods include molding, milling, or even 3D printing. In the case of such 3D printing, it typically uses a metal powder whose shaping is ensured by a laser of suitable power.

Likewise, welding or crimping can be carried out using techniques well known to those skilled in the art. In the case where 3) at least one device is made of titanium or a titanium alloy, the welding of its various components can then in particular be carried out by laser, by friction or by electron bombardment.

In connection with the 4) at least one complementary piece of the device ensuring the maintenance of the cover membrane (6) and which makes it easier for the practitioner to grip said device, this 4) at least one complementary piece therefore allows to facilitate the positioning of said 3) at least one device on the 1) at least one anchor foot in the patient's mouth by the practitioner.

Typically, this complementary piece, which constitutes a gripping means takes the form of a sleeve, which can be made of plastic, and which clips to the central part of the 3) at least one cover piece (8), typically in a housing located within this central part.

The examples which follow are provided for illustration purposes and cannot limit the scope of the present invention.

Bone Regeneration of a Bone Defect of the Jaw with a View to the Placement of Dental Implants

In the context of a bone defect in the jaw, it is necessary to allow regeneration of this bone defect before being able to consider the placement of dental implants.

To do this, an incision in the gingiva is made in order to access this bone defect in the jaw. This incision is made by elevating the gingiva flap resulting from the incision.

The practitioner can then drill into the jaw bone, ideally at or near the bone defect. The practitioner then installs an anchor foot in the drilling carried out previously.

Once this installation has been carried out, the practitioner fills the bone defect with a bone regeneration substitute.

FIG. 1 provides a sectional view of the gingiva incised in order to access the bone defect with the elevation of a gum flap (L) of gingiva following the incision. We also observe the anchor foot (3) positioned within the drilling which was carried out by the practitioner in the bone (1) of the patient's jaw. Around the anchor foot (3), we visualize the filling of the bone defect with the bone regeneration substitute (11). The anchor foot (3) is overlooked by the device (2) for holding the bone regeneration substrate (11) with which it is associated by a screw (9). Note that, in this section, the cover membrane (6) is positioned on the device (2).

FIG. 2 shows a sectional view this time of the bone regeneration system presented in FIG. 1, in which we observe the anchor foot (3) and the device (2) with its elongated lateral extensions (7a, 7b). This anchor foot (3) and this device (2) are associated by screwing the external thread (9a) of a screw (9) into the thread (3b) of the internal cavity (3a) of the anchor foot (3). Indeed, an internal cavity (4a) of the device (2) for holding the bone regeneration substitute (11) allows the passage of this screw (9) but not of its head (9b).

FIGS. 3 to 6 show schematic views from different angles of a device (2) for maintaining the bone regeneration substitute. In these, we can observe the device (2) with its external pillar (4), which external pillar (4) is equipped with anti-rotational means taking the form of a Morse cone so as to limit the movements of the device (2) once its external pillar (4) has been placed within the complementary cavity of the anchor foot (see FIG. 2). This external pillar (4) is topped by an intermediate pillar (5) of conical shape so as to facilitate its passage within the gum. Above the intermediate pillar (5), the cover piece (8) is positioned with its cylindrical central part (12), which central part (12) is an extension of the intermediate pillar (5). The elongated lateral extensions (7a, 7b) of the cover piece (8) comprise two elongated palatal lateral extensions (7b), the shortest, and three elongated vestibular lateral extensions (7a), the longest. These latter elongated vestibular lateral extensions (7a) each further comprise a breaking point (10) which facilitates the adjustment of the device (2) by the practitioner so as to ensure a safe and comfortable placement adapted to the patient. These same elongated vestibular lateral extensions (7a) also include two primers (13), positioned along its length, to facilitate the folding by the practitioner of these extensions and the adjustment of the device to the morphology of the patient.

FIG. 4 shows an aerial perspective of the cover part (8) of the device (2), which allows a better understanding of the specific arrangement of its components. In this, we observe the elongated lateral extensions (7a, 7b) in the extension of the central part (12) whose axis intersects perpendicularly the central axis (AA) (visible in FIG. 6 also). We also observe in this view, the internal cavity (4a) passing through the cover piece (8), but also (not visible in this view) the external pillar and the intermediate pillar.

We claim:

1. A device (2) intended to ensure the maintenance of a bone regeneration substitute over a bone defect in a jaw bone (1) of a patient comprising:
a. at least one external pillar (4) on its face directed towards the bone (1), which is intended to be fixed in the anchor foot (3), which external pillar (4) comprises anti-rotational means making it possible to inhibit the relative movements of the external pillar (4) relative to the anchor foot (3) once the external pillar (4) placed in the anchor foot (3); and
b. at least one cover piece (8) on its face opposite the bone (1), which is placed on the bone regeneration substitute (11) and, optionally, on a cover membrane (6), so as to delimit the volume of bone regeneration at said bone defect site in combination with said bone regeneration substitute (11) and, optionally, said cover membrane (6), which cover piece (8) forms a single piece with the external pillar (4) and presents:
i. a central part (12), in the continuation of the external pillar (4), and
ii. at least 2 flexible elongated lateral extensions (7a, 7b) in the continuation of this i) central part (12), which does not comprise any orifice intended for positioning of a fixing screw, and whose axis intersects perpendicularly the central axis (AA), corresponding to the axis crossing the central part (12).

2. The device (2) of claim 1, wherein the central part (12) has two distinct halves for facilitating their differentiation by the practitioner of the vestibular side and the palatal side, with a half of the central part (12) facing the vestibular side when placed by the practitioner (i.e., towards the outside the mouth) in the continuation of which the flexible elongated lateral extensions, or flexible elongated vestibular lateral extensions (7a), have a greater length than the flexible elongated lateral extensions in the continuation of the other half of the central part (12) which faces the palatal side when placed by the practitioner (i.e. towards the palate) or flexible elongated palatal lateral extensions (7b).

3. The device (2) of claim 2, wherein the flexible elongated vestibular lateral extensions (7a) have a length of between 4 and 10 mm, and the flexible elongated palatal lateral extensions (7b) have a length of between 3 and 6 mm.

4. The device (2) of claim 2, wherein said device (2) comprises 2 to 5 flexible elongated vestibular lateral extensions (7a), and 1 to 4 flexible elongated palatal lateral extensions (7b)

5. The device (2) of claim 1, wherein each flexible elongated lateral extension (7a, 7b) has one or more predetermined breaking points (10) positioned along its length.

6. The device (2) of claim 1, wherein each flexible elongated lateral extension (7a, 7b) has one or more comprises one or more primers (13) facilitating its folding, and which are positioned along its length.

7. The device (2) of claim 1, wherein said device (2) comprises an intermediate pillar (5) between the external pillar (4) and the cover piece (8), and wherein the external pillar (4), the intermediate pillar (5), and the cover piece (8) form a single piece.

8. The device (2) of claim 7, wherein the height of said intermediate pillar (5) is in the range from 0.5 to 5 mm.

9. The device (2) of claim 1, wherein the cover piece (8) has a thickness of between 0.3 and 3 mm and the flexible elongated lateral extensions (7a, 7b) have a thickness of between 0.1 and 1.5 mm.

10. The device (2) of claim 7, wherein the central part (12) is circular.

11. The device (2) of claim 7, wherein said device (2) is made of titanium.

12. A bone regeneration system for a bone defect site in a jaw bone (1) of a patient with a bone regeneration substitute (11) allowing the filling of said bone defect, which comprises:
1. at least one anchor foot (3) suitable for endosteal installation in the jaw bone (1);
2. at least one device (2) intended to ensure the maintenance of a bone regeneration substitute over a bone defect in a jaw bone (1) of a patient comprising:
a. at least one external pillar (4) on its face directed towards the bone (1), which is intended to be fixed in the anchor foot (3), which external pillar (4) comprises anti-rotational means making it possible to inhibit the relative movements of the external pillar (4) relative to the anchor foot (3) once the external pillar (4) placed in the anchor foot (3); and
b. at least one cover piece (8) on its face opposite the bone (1), which is placed on the bone regeneration substitute (11) and, optionally, on a cover membrane (6), so as to delimit the volume of bone regeneration at said bone defect site in combination with said bone regeneration piece (8) forms a single piece with the external pillar (4) and presents:
i. a central part (12), in the continuation of the external pillar (4), and
ii. at least 2 flexible elongated lateral extensions (7a, 7b) in the continuation of this i) central part (12), which does not comprise any orifice intended for positioning of a fixing screw, and whose axis intersects perpendicularly the central axis (AA), corresponding to the axis crossing the central part (12) and the anchor foot (3).

13. The bone regeneration system of claim 12, wherein the a central part (12) has two distinct halves for facilitating their differentiation by the practitioner of the vestibular side and the palatal side, with a half of the central part (12) facing the vestibular side when placed by the practitioner (i.e., towards the outside the mouth) in the continuation of which the flexible elongated lateral extensions, or flexible elongated vestibular lateral extensions (7a), have a greater length than the flexible elongated lateral extensions in the continuation of the other half of the central part (12) which faces the palatal side when placed by the practitioner (i.e. towards the palate) or flexible elongated palatal lateral extensions (7b).

14. The bone regeneration system of claim 13, wherein the flexible elongated vestibular lateral extensions (7a) have a length of between 4 and 10 mm, and the flexible elongated palatal lateral extensions (7b) have a length of between 3 and 6 mm.

15. The bone regeneration system of claim 13, wherein the device (2) comprises 2 to 5 flexible elongated vestibular lateral extensions (7a), and 1 to 4 flexible elongated palatal lateral extensions (7b)

16. The bone regeneration system of claim 12, wherein each flexible elongated lateral extension (7a, 7b) has one or more predetermined breaking points (10) positioned along its length.

17. The bone regeneration system of claim 12, wherein each flexible elongated lateral extension (7a, 7b) has one or more comprises one or more primers (13) facilitating its folding, and which are positioned along its length.

18. The bone regeneration system of claim 12, wherein the device (2) comprises an intermediate pillar (5) between the external pillar (4) and the cover piece (8), and wherein the external pillar (4), the intermediate pillar (5), and the cover piece (8) form a single piece.

19. The bone regeneration system of claim 12, which said bone regeneration system further comprises:
3. at least one cover membrane (6), which is of resorbable or non-resorbable nature, and whose surface makes it possible to cover at least the entire bone defect.

20. The bone regeneration system of claim 12, which said bone regeneration system further comprises:
4. at least one piece facilitating the gripping of the device (2) by the practitioner, taking the form of a sleeve, and which clips to the central part (12) of the device (2) in a complementary housing located within this central part (20).

21. The bone regeneration system of claim 12, wherein said bone regeneration system further comprises at least one screw (9) having a head (9b) and an external thread (9a) at its distal end; wherein the external pillar (4) of the device (2) has an internal cavity (4a) allowing the passage of said screw (9) but not of its head (9b); and wherein the anchor foot (3) has a cavity (3a) in which is provided a tapping (3b), which can cooperate with the thread (9a) of said screw (9) to allow the assembly of the at least one anchor foot (3) and of the at least one device (2).

22. A bone regeneration kit for a bone defect site in a jaw bone (1) of a patient which comprises:
1. a bone regeneration substitute (11) allowing the filling of the bone defect;
2. at least one anchor foot (3) suitable for endosteal installation in the jaw bone (1);
3. at least one device (2) intended to ensure the maintenance of a bone regeneration substitute over a bone defect in a jaw bone (1) of a patient comprising:
a. at least one external pillar (4) on its face directed towards the bone (1), which is intended to be fixed in the anchor foot (3), which external pillar (4) comprises anti-rotational means making it possible to inhibit the relative movements of the external pillar (4) relative to the anchor foot (3) once the external pillar (4) placed in the anchor foot (3); and
b. at least one cover piece (8) on its face opposite the bone (1), which is placed on the bone regeneration substitute (11) and, optionally, on a cover membrane (6), so as to delimit the volume of bone regeneration at said bone defect site in combination with said bone regeneration piece (8) forms a single piece with the external pillar (4) and presents:
i. a central part (12), in the continuation of the external pillar (4), and
ii. at least 2 flexible elongated lateral extensions (7a, 7b) in the continuation of this i) central part (12), which does not comprise any orifice intended for positioning of a fixing screw, and whose axis intersects perpendicularly the central axis (AA), corresponding to the axis crossing the central part (12) and the anchor foot (3).

23. The bone regeneration kit of claim 22, wherein the a central part (12) has two distinct halves for facilitating their differentiation by the practitioner of the vestibular side and the palatal side, with a half of the central part (12) facing the vestibular side when placed by the practitioner (i.e., towards the outside the mouth) in the continuation of which the flexible elongated lateral extensions, or flexible elongated vestibular lateral extensions (7a), have a greater length than the flexible elongated lateral extensions in the continuation of the other half of the central part (12) which faces the palatal side when placed by the practitioner (i.e. towards the palate) or flexible elongated palatal lateral extensions (7b).

24. The bone regeneration kit of claim 23, wherein the flexible elongated vestibular lateral extensions (7a) have a length of between 4 and 10 mm, and the flexible elongated palatal lateral extensions (7b) have a length of between 3 and 6 mm.

25. The bone regeneration kit of claim 23, wherein the device (2) comprises 2 to 5 flexible elongated vestibular lateral extensions (7a), and 1 to 4 flexible elongated palatal lateral extensions (7b)

26. The bone regeneration kit of claim 22, wherein each flexible elongated lateral extension (7a, 7b) has one or more predetermined breaking points (10) positioned along its length.

27. The bone regeneration kit of claim 22, wherein each flexible elongated lateral extension (7a, 7b) has one or more comprises one or more primers (13) facilitating its folding, and which are positioned along its length.

28. The bone regeneration kit of claim 22, wherein the device (2) comprises an intermediate pillar (5) between the external pillar (4) and the cover piece (8), and wherein the external pillar (4), the intermediate pillar (5), and the cover piece (8) form a single piece.

29. The bone regeneration kit of claim 22, which said bone regeneration kit further comprises:
4. at least one cover membrane (6), which is of resorbable or non-resorbable nature, and whose surface makes it possible to cover at least the entire bone defect.

30. The bone regeneration kit of claim 22, which said bone regeneration kit further comprises:
5. at least one piece facilitating the gripping of the device (2) by the practitioner, taking the form of a sleeve, and which clips to the central part (12) of the device (2) in a complementary housing located within this central part (2).

31. The bone regeneration kit of claim 22, wherein said bone regeneration kit further comprises at least one screw (9) having a head (9b) and an external thread (9a) at its distal end; wherein the external pillar (4) of the device (2) has an internal cavity (4a) allowing the passage of said at least one screw (9) but not of its head (9b); and wherein the anchor foot (3) has a cavity (3a) in which is provided a tapping (3b) which can cooperate with the thread (9a) of said at least one screw (9) to allow the assembly of the at least one anchor foot (3) and of the at least one device (2).

32. A method of bone regeneration for a bone defect site of moderate size in a jaw bone (1) of a patient comprising the steps of:
i. placement of an anchor foot (3) within the patient's jaw bone (1) at or near the bone defect;
ii. filling of the bone defect with a bone regeneration substitute (11);
iii. eventually positioning, on this bone regeneration substitute (11), a cover membrane (6); and
iv. placement, preferably on this cover membrane (6) if positioned, a device (2) intended to ensure the maintenance of the bone regeneration substitute and, if present, the cover membrane (6);
Wherein the device (2) comprises:
a. at least one external pillar (4) on its face directed towards the bone (1), which is intended to be fixed in the anchor foot (3), which external pillar (4) comprises anti-rotational means making it possible to inhibit the relative movements of the external pillar (4) relative to the anchor foot (3) once the external pillar (4) placed in the anchor foot (3); and
b. at least one cover piece (8) on its face opposite the bone (1), which is placed on the bone regeneration substitute (11) and, optionally, on a cover membrane (6), so as to delimit the volume of bone regeneration at said bone defect site in combination with said bone regeneration substitute (11) and, optionally, said cover membrane (6), which cover piece (8) forms a single piece with the external pillar (4) and presents:
i. a central part (12), in the continuation of the external pillar (4), and
ii. at least 2 flexible elongated lateral extensions (7a, 7b) in the continuation of this i) central part (12), which does not comprise any orifice intended for positioning of a fixing screw, and whose axis intersects perpendicularly the central axis (AA), corresponding to the axis crossing the central part (12).

33. The method of claim 32, wherein the step iv. of placement of the device (2) by the practitioner if facilitated by using a device (2), whose central part (12) has two distinct halves differentiating the vestibular side and the palatal side, with a half of the central part (12) facing the vestibular side when placed by the practitioner (i.e., towards the outside the mouth) in the continuation of which the flexible elongated lateral extensions, or flexible elongated vestibular lateral extensions (7a), have a greater length than the flexible elongated lateral extensions in the continuation of the other half of the central part (12) which faces the palatal side when placed by the practitioner (i.e. towards the palate) or flexible elongated palatal lateral extensions (7b).

34. The method of claim 33, wherein the flexible elongated vestibular lateral extensions (7a) of the device (2) have a length of between 4 and 10 mm, and the flexible elongated palatal lateral extensions (7b) have a length of between 3 and 6 mm.

35. The method of claim 33, wherein the device (2) comprises 2 to 5 flexible elongated vestibular lateral extensions (7a), and 1 to 4 flexible elongated palatal lateral extensions (7b).

36. The method of claim 32, wherein, and for the step iv. of placement of the device (2), the practitioner positions the at least one device (2) on the at least one anchor foot (3) in the patient's mouth by griping at least one piece, taking the form of a sleeve, and which clips to the central part (12) of the device (2) in a complementary housing located within this central part (12).

37. The method of claim 32, wherein, and for the step iv. of placement of the device (2), the practitioner uses one or more predetermined breaking points (10) positioned along the length of each flexible elongated lateral extension (7a, 7b) of the device (2) so as to shorten all or part of these elongated lateral extensions (7a, 7b) and to adapt them to the patient's morphology.

38. The method of claim 32, wherein, and for the step iv. of placement of the device (2), the practitioner uses one or more primers (13) positioned along the length of each flexible elongated lateral extension (7a, 7b) of the device (2) for facilitating its folding and adaption to the morphology of the patient.

39. The method of claim 32, wherein the device (2) comprises an intermediate pillar (5) between the external pillar (4) and the cover piece (8), and wherein the external pillar (4), the intermediate pillar (5), and the cover piece (8) form a single piece.

40. The method of claim 38, wherein the height of the intermediate pillar (5) of the device (2) is in the range from 0.5 to 5 mm.

41. The method of claim 38, wherein the cover piece (8) of the device (2) has a thickness of between 0.3 and 3 mm and the flexible elongated lateral extensions (7a, 7b) of the device (2) have a thickness of between 0.1 and 1.5 mm.

42. The method of claim 32, wherein the central part (12) of the device (2) is circular.

43. The method of claim 32, wherein said device (2) of the device (2) is made of titanium.
